(19) United States
(12) Patent Application Publication
(10) Pub. No.: US 2024/0033502 A1
(43) Pub. Date: Feb. 1, 2024

(54) FLEXIBLE SELF-POWERED MATERIALS FOR ON DEMAND GENERATION OF HYDROGEN PEROXIDE

(71) Applicant: Wesbot, LLC, Columbus, OH (US)

(72) Inventors: Anne Co, Columbus, OH (US); Wesley Thio, Columbus, OH (US)

(21) Appl. No.: 17/874,198

(22) Filed: Jul. 26, 2022

(51) Int. Cl. A61N 1/04; A61L 2/00; A61L 2/18; A61L 2/26; A61K 33/40; A61C 19/06; A61K 8/02; A61Q 11/00; A61P 31/04; A61K 8/22; C25B 1/30; A47L 13/17; D03D 15/533; D06M 11/83; D03D 1/00; D06M 15/277

(52) U.S. Cl. CPC A61N 1/0496; A61L 2/0088; A61L 2/186; A61L 2/26; A61N 1/0468; A61K 33/40; A61C 19/066; A61K 8/0208; A61Q 11/00; A61P 31/04; A61K 8/22; C25B 1/30; A47L 13/17; D03D 15/533; D06M 11/83; D03D 1/0023; D03D 1/00; D06M 15/277; A61L 2101/02

(57) ABSTRACT

Disclosed are materials/systems and methods that are selective for the formation of hydrogen peroxide (H₂O₂) via an electrochemical reaction. The materials/systems comprise a substrate and at least one pair of electrodes positioned on or within the substrate, wherein each pair of electrodes comprises an anode and a cathode. At least one of the anode and the cathode comprise a catalyst that can form hydrogen peroxide (H₂O₂). The catalyst can comprise a nanoporous Cu catalyst.

### BACKGROUND OF THE INVENTION

Hydrogen peroxide is used in the medical and dental fields for a number of applications, including disinfecting wounds and whitening teeth. However, hydrogen peroxide is prone to decompose and lose its disinfectant and stain removing characteristics. Currently, reapplication of hydrogen peroxide is the primary solution to maintain the disinfecting and stain removing characteristics of hydrogen peroxide over an extended period. There is a need for a solution that more effectively administers hydrogen peroxide. What are needed are new methods of producing hydrogen peroxide on demand. For example, methods of producing hydrogen peroxide directly from hydrogen and oxygen on-site, reducing the tendency for hydrogen peroxide to decompose before use are needed. The systems and methods disclosed herein address these and other needs.

### SUMMARY OF THE INVENTION

Disclosed herein are systems that electrochemically produce hydrogen peroxide and methods of using the systems in one or more applications (e.g., wound cleaning, tooth whitening, or stain removal). The systems can include at least one pair of electrodes positioned on or within a substrate, wherein each pair of electrodes comprises an anode and a cathode. At least one of the anode and the cathode comprise a catalyst that is selective for the formation of hydrogen peroxide (H₂O₂) via an electrochemical reaction. The specific substrate used in the systems disclosed herein may depend on the end use of the systems. For example, the at least one pair of electrodes can be affixed to or otherwise embedded, such as by printing, to a substrate (e.g., a biocompatible fabric substrate or a cleaning wipe) or to a substrate that serves as an external surface of a tooth whitening system following application to a tooth surface.

The catalyst provided in the systems that is selective for the formation of hydrogen can comprise nanoparticles having an average particle size of from 10 nm to 500 nm, as determined by scanning electron microscopy (SEM). In some embodiments, the catalyst comprises copper, copper compounds, gold, gold compounds, iron, iron compounds, vanadium compounds, graphite, porous carbon, or a metal oxide such as tungsten oxide or cerium oxide, or a combination thereof. For example, the catalyst can comprise a nanoporous Cu catalyst; e.g., a nanoporous Cu-M catalyst, wherein M is a metal chosen from Pt, Ir, Pd, Ag, Au, Rh, Ru, Zn, Sn, Ni, Fe, Re, Ga, In, Cd, Tl, and Ti; or a combination thereof. In some examples, the catalyst is a nanoporous Cu catalyst.

When contacted with an aqueous fluid, the catalyst electrochemically oxidizes water to produce hydrogen peroxide, or reduces oxygen to produce hydrogen peroxide. The aqueous fluid, in one aspect is a conductive liquid, such as perspiration, wound exudate, saline, saliva, or water. In some embodiments, the aqueous fluid has a salinity 0.25 pph or greater (about 44 mmol or greater), such as 0.6 pph or greater, for example, from 0.25 pph to 3 pph, or from 0.6 pph to 3 pph.

The systems that electrochemically produce hydrogen peroxide can further comprise a power source having an output electrically coupled to the at least one electrode pair, wherein the power source comprises one or more cells. In some embodiments, the power source comprises a plurality of cells. The plurality of cells can be connected using flexible connectors in electrical parallel to increase current, thereby increasing the amount of hydrogen peroxide generated.

In another aspect provides kits comprising the systems disclosed herein.

In other aspects, there are provided methods of treating or preventing a bacterial infection in a wound; methods of removing stain from a surface; and/or tooth whitening methods, using the hydrogen peroxide generating systems disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary schematic diagram showing a battery hydrogen peroxide generating system, such as an integrated self-powered hydrogen peroxide generating fabric.

FIG. 2 illustrates an exemplary image showing a printed catalyst on a soaked fabric generating hydrogen peroxide (white region around dot) when powered. The amount of hydrogen peroxide generated is dependent on the current and time run (e.g., 1 mA for 5 minutes generates 5 μmol on one dot).

FIG. 3A/B show some general principles of hydrogen peroxide generation. FIG. 3A is a graph showing how far the voltage shifts from OCP on application of positive and negative currents. FIG. 3B shows hydrogen peroxide can be generated through oxidation or reduction using a copper catalyst. The determined oxidation (current out of Cu) will require less power for the same generated hydrogen peroxide.

FIG. 4 provides an image showing an exemplary soft and flexible battery with a Zn electrode 8 and Ag₂O electrode 9 powered by sweat. The battery in NaCl or buffer has comparable capacity to a standard commercial NaOH battery.

FIG. 5 provides an exemplary image showing the scalability of a soft and flexible battery. The speed of hydrogen peroxide generation can be controlled through a number of batteries. The time the device runs can be controlled by adjusting battery capacity. One battery (1 Volt) draws 0.1 mA and delivers 0.52 μM/sec, which is safe for human use.

FIGS. 6A and 6B show destaining of eggs. FIG. 6A is an image showing eggs initially stained with coffee and treated with a H₂O₂ concentration equivalent to commercial products. The eggs were then crushed and analyzed using UV-VIS. FIG. 6B is a graph showing UV-VIS of crushed eggshells with CaCO₃ background (R %). The lighter eggshells are more reflective compared to darker eggshells.

FIG. 7 shows disinfection of E. coli (BW25113) bacteria using hydrogen peroxide and electric current. Bacteria were counted by Colony Forming Units (CFU). The H₂O₂ was generated independently then mixed with bacteria and plated. CFU was counted to find % CFU/μmol H₂O₂.

FIG. 8A/B show application of the systems to disinfecting and wound healing. FIG. 8A shows a filter paper soaked with generated 0.75 M hydrogen peroxide and disinfection in region of clearance. FIG. 8B shows a filter paper soaked in PBS solution with no hydrogen peroxide. No region of clearance was observed.

### DETAILED DESCRIPTION OF THE INVENTION

Reference in the specification to “a specific embodiment” or a similar expression means that a particular feature, structure, or characteristic described in connection with the specific embodiment is included in at least one specific embodiment of the present invention. Therefore, in this specification, the appearance of the terms “in a specific embodiment” and similar expressions does not necessarily refer to the same specific embodiment.

Provided herein are systems and methods for electrochemically producing hydrogen peroxide. The systems disclosed herein for electrochemically producing hydrogen peroxide comprise at least one pair of electrodes positioned on or within a substrate, wherein each pair of electrodes comprises an anode and a cathode. At least one of the anodes and the cathodes comprise a catalyst that is selective for the production of hydrogen peroxide (H₂O₂) via an electrochemical reaction.

The catalysts described herein are selective for producing hydrogen peroxide. In some embodiments, the catalyst comprises copper, copper compounds, gold, gold compounds, iron, iron compounds, vanadium compounds, graphite, porous carbon, or a metal oxide such as tungsten oxide or cerium oxide, or a combination thereof. In certain embodiments, the catalyst comprises a nanoporous Cu catalyst; such as a nanoporous Cu-M catalyst, where M is a metal chosen from Pt, Ir, Pd, Ag, Au, Rh, Ru, Zn, Sn, Ni, Fe, Re, Ga, In, Cd, Tl, and Ti; or a combination thereof.

The nanoporous Cu catalyst can be a nanoporous, open-cell copper foam. Nanoporous, open-cell copper foams are known in the art, and can be prepared from alloys of copper and a second, less noble metal (e.g., aluminum, zinc, magnesium, tin, etc.). The second, less noble metal can be selectively removed, for example by etching the alloy (a process also referred to as selective leaching or dealloying), to provide a porous copper material. This process involves contacting an alloy of copper and a second, less noble metal with an etchant for a period of time effective to selectively leach the second, less noble metal from the copper and form a porous copper support. An appropriate etchant can be selected in view of the identity of the second, less noble metal. For example, in some embodiments, the nanoporous, open-cell copper foam can be prepared by etching CuAl alloy (e.g., by contacting the CuAl alloy with a suitable etchant, for example a base such as aqueous sodium hydroxide, for a period of time effective to selectively leach the aluminum from the copper) to form a nanoporous, open-cell copper foam.

The relative amounts of copper and the second, less noble metal in the alloy used to form the nanoporous, open-cell copper foam can be varied in order to influence the properties of the resulting nanoporous, open-cell copper foam (and thus the resulting catalytic properties of the material). In some embodiments, the alloy of copper and a second, less noble metal (e.g., aluminum) comprise at least 10 atomic percent (at %) copper (e.g., at least 15 at % copper, at least 20 at % copper, at least 25 at % copper, at least 30 at % copper, at least 35 at % copper, at least 40 at % copper, or at least 45 at % copper). In some embodiments, the alloy of copper and a second, less noble metal (e.g., aluminum) comprise 50 at % or less copper (e.g., 45 at % or less copper, 40 at % or less copper, 35 at % or less copper, 30 at % or less copper, 25 at % or less copper, 20 at % or less copper, or 15 at % or less copper). In some embodiments, the alloy of copper and a second, less noble metal (e.g., aluminum) comprise at least 50 at % of the second, less noble metal (e.g., at least 55 at % of the second, less noble metal, at least 60 at % of the second, less noble metal, at least 65 at % of the second, less noble metal, at least 70 at % of the second, less noble metal, at least 75 at % of the second, less noble metal, at least 80 at % of the second, less noble metal, or at least 85 at % of the second, less noble metal). In some embodiments, the alloy of copper and a second, less noble metal (e.g., aluminum) comprise 90 at % or less of the second, less noble metal (e.g., 85 at % or less of the second, less noble metal, 80 at % or less of the second, less noble metal, 75 at % or less of the second, less noble metal, 70 at % or less of the second, less noble metal, 65 at % or less of the second, less noble metal, 60 at % or less of the second, less noble metal, or 55 at % or less of the second, less noble metal).

The relative amounts of copper and the second, less noble metal (e.g., aluminum) in the alloy (e.g., CuAl) used to form the nanoporous, open-cell copper foam can range from any of the minimum values described above to any of the maximum values described above. In some embodiments, the alloy of copper and a second, less noble metal (e.g., aluminum) comprises from 10 to 50 at % copper and from 50 to 90 at % of the second, less noble metal (e.g., Al). In certain embodiments, the alloy used to form the nanoporous, open-cell copper foam is a CuAl alloy that comprises from 10 to 50 at % copper and from 50 to 90 at % aluminum (e.g., from 10 to 30 at % copper and from 70 to 90 at % aluminum).

The nanoporous Cu-M catalyst can be a nanoporous, open-cell Cu-M alloy foam. Nanoporous, open-cell Cu-M alloy foams can be prepared by galvanically depositing a metal M (e.g., Pt, Ir, Pd, Ag, Au, Rh, Ru, Zn, Sn, Ni, Fe, Re, Ga, In, Cd, Tl, and Ti) on a nanoporous, open-cell copper foam to form the nanoporous, open-cell Cu-M alloy foam. Methods for producing the nanoporous Cu-M catalyst can comprise galvanically depositing a catalytically effective amount of a desired metal (M) on a nanoporous, open-cell copper foam (e.g., at a temperature greater than 5° C.) to form a Cu-M precursor catalyst; and conditioning the Cu-M precursor catalyst to form the nanoporous Cu-M catalyst.

Galvanic deposition involves contacting the nanoporous, open-cell copper foam with a solution (e.g., an aqueous solution) comprising an M-containing species (e.g., a Pt, Ir, Pd, Ag, Au, Rh, Ru, Zn, Sn, Ni, Fe, Re, Ga, In, Cd, Tl, or Ti-containing species). The M-containing species comprise a suitable metal complex that participates in a spontaneous galvanic-reaction with the copper in the nanoporous, open-cell copper foam. By way of example, in the case of the galvanic deposition of Pt, the Pt-containing species may comprise a platinum metal complex that participates in a spontaneous galvanic-reaction with the copper in the porous copper support, such as PtCl₄²⁻, PtCl₆²⁻, or combinations thereof.

In some embodiments, the nanoporous, open-cell copper foam is disposed on a surface (e.g., the surface of an electrode) in contact with the solution comprising the M-containing species during galvanic deposition. In certain embodiments, the surface comprising the nanoporous, open-cell copper foam is rotated during galvanic deposition. The surface can be rotated at a rate effective to induce a laminar flow of the solution comprising the M-containing species towards and across the surface on which the nanoporous, open-cell copper foam is disposed during galvanic deposition. This can drive uniform deposition of the metal on the nanoporous, open-cell copper foam. In certain embodiments, the surface is rotated at a rate of from 250 rpm to 2000 rpm (e.g., from 250 rpm to 1500 rpm, or from 250 rpm to 750 rpm).

The galvanic deposition can be performed at varying temperatures to provide nanoporous Cu-M catalysts having the desired properties for a particular catalytic application. In some embodiments, the galvanic deposition is performed at a temperature greater than 5° C. (e.g., at least 10° C., at least 15° C., at least 20° C., at least 25° C., at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., at least 100° C., at least 110° C., at least 120° C., at least 130° C., at least 140° C., at least 150° C., at least 160° C., at least 170° C., at least 180° C., or at least 190° C.). In some embodiments, the galvanic deposition is performed at a temperature of 200° C. or less (e.g., 190° C. or less, 180° C. or less, 170° C. or less, 160° C. or less, 150° C. or less, 140° C. or less, 130° C. or less, 120° C. or less, 110° C. or less, 100° C. or less, 95° C. or less, 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 20° C. or less, 15° C. or less, or 10° C. or less).

The galvanic deposition can be performed at a temperature ranging from any of the minimum temperature values described above to any of the maximum temperatures described above. In some embodiments, the metal is galvanically deposited at a temperature of from 5° C. to 200° C. (e.g., from 5° C. to 170° C., from 5° C. to 150° C., from 5° C. to 120° C., from 5° C. to 90° C., from 5° C. to 90° C., from 25° C. to 90° C., from 5° C. to 60° C., or from 25° C. to 60° C.).

The galvanic deposition can be performed for varying periods of time, so as to provide nanoporous Cu-M catalysts having a molar ratio of Cu:M desired for use in a particular catalytic application. For example, the nanoporous, open-cell copper foam is maintained in contact with the solution comprising the M-containing species for a period of time effective to form a nanoporous Cu-M catalyst having desired a molar ratio of Cu:M.

The molar ratio of Cu:M in the nanoporous Cu-M catalyst can be determined by Inductively Coupled Plasma-Mass Spectroscopy (ICP-MS). In some embodiments, molar ratio of Cu:M in the nanoporous Cu-M catalyst is at least 1:2 (e.g., at least 1:1, at least 1.25:1, at least 1.5:1, at least 1.6:1, at least 1.7:1, at least 1.8:1, at least 1.9:1, at least 2:1, at least 2.1:1, at least 2.2:1, at least 2.3:1, at least 2.4:1, at least 2.5:1, at least 5:1, at least 10:1, at least 25:1, at least 50:1 , at least 100:1, at least 150:1, at least 200:1, or at least 250:1). In some embodiments, molar ratio of Cu:M in the nanoporous Cu-M catalyst is 500:1 or less (e.g., 250:1 or less, 200:1 or less, 150:1 or less, 100:1 or less, 50:1 or less, 25:1 or less, 10:1 or less, 5:1 or less, 2.5:1 or less, 2.4:1 or less, 2.3:1 or less, 2.2:1 or less, 2.1:1 or less, 2:1 or less, 1.9:1 or less, 1.8:1 or less, 1.7:1 or less, 1.6:1 or less, 1.5:1 or less, 1.25:1 or less, or 1:1 or less).

The molar ratio of Cu:M in the nanoporous Cu-M catalyst can range from any of the minimum ratios described above to any of the maximum ratios described above. In some embodiments, the molar ratio of Cu:M in the nanoporous Cu-M catalyst, as determined by ICP-MS, ranges from 1:2 to 500:1 (e.g., from 1:2 to 250:1; from 1:1 to 500:1; from 1:1 to 250:1; from 5:1 to 500:1; from 10:1 to 500:1; from 0.5:1 to 2.5:1, from 1:1 to 2.5:1, or from 1.5:1 to 2.2:1).

Following galvanic deposition, the Cu-M precursor catalyst can be conditioned to form the nanoporous Cu-M catalyst. Conditioning can involve electrochemical dealloying of the Cu-M precursor catalyst to form the nanoporous Cu-M catalyst. In some embodiments, the Cu-M precursor catalyst is conditioned by repeated electrochemical cycling (e.g., 50 cycles) of the Cu-M precursor catalyst between 0.5 V and 1.2 V at 25° C. in N₂-saturated 0.1 M HClO₄ to dealloy/stabilize the catalyst.

In some embodiments, the catalyst (e.g., the nanoporous Cu catalyst; the nanoporous Cu-M catalyst, where M is a metal chosen from Pt, Ir, Pd, Ag, Au, Rh, Ru, Zn, Sn, Ni, Fe, Re, Ga, In, Cd, Tl, and Ti; or a combination thereof) is processed to reduce the particle size of the catalyst prior to use in conjunction with the methods described herein. In some embodiments, the catalyst is formed into nanoparticles prior to use in conjunction with the methods described herein.

The catalyst may be formed into nanoparticles prior to use in conjunction with the methods described herein using any suitable method known in the art. The nanoparticles formed by the process can be spherical or non-spherical in shape. In certain embodiments, the nanoparticles are discrete, spherical nanoparticles. In some embodiments, the population of nanoparticles formed by this process is monodisperse. In some embodiments, the nanoparticles optionally comprise nanopores. In some embodiments, the nanopores interconnect, so as to form a network of nanopores spanning the nanoparticles.

“Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

“Mean particle size” or “average particle size”, are used interchangeably herein, and generally refer to the statistical mean particle size of the particles in a population of nanoparticles. The diameter of an essentially spherical particle can refer to the physical diameter of the spherical particle. The diameter of a non-spherical nanoparticle can refer to the largest linear distance between two points on the surface of the nanoparticle. Mean particle size can be measured using methods known in the art, such as evaluation by scanning electron microscopy.

In some embodiments, the catalyst comprises nanoparticles having an average particle size, as measured by scanning electron microscopy (SEM), of at least 10 nm (e.g., at least 15 nm, at least 20 nm, at least 25 nm, at least 30 nm, at least 35 nm, at least 40 nm, at least 45 nm, at least 50 nm, at least 55 nm, at least 60 nm, at least 65 nm, at least 70 nm, at least 75 nm, at least 80 nm, at least 85 nm, at least 90 nm, at least 95 nm, at least 100 nm, at least 150 nm, at least 200 nm, at least 250 nm, at least 300 nm, at least 350 nm, at least 400 nm, or at least 450 nm). In some embodiments, the catalyst comprise nanoparticles having an average particle size, as measured by SEM, of 500 nm or less (e.g., 450 nm or less, 400 nm or less, 350 nm or less, 300 nm or less, 250 nm or less, 200 nm or less, 150 nm or less, 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, 25 nm or less, 20 nm or less, or 15 nm or less).

The catalyst can comprise nanoparticles having an average particle size, as measured by SEM, ranging from any of the minimum values described above to any of the maximum values described above. In some embodiments, the catalyst comprise nanoparticles having an average particle size, as measured by SEM, of from 10 nm to 500 nm (e.g., from 10 nm to 250 nm, from 10 nm to 150 nm, from 20 nm to 100 nm, from 20 nm to 80 nm, or from 80 nm to 100 nm, from 10 nm to 80 nm, from 25 nm to 80 nm, or from 50 nm to 80 nm).

In some embodiments, the catalyst has a specific surface area of at least 5 m²/g, as measured using the Brunauer-Emmett-Teller (BET) method (e.g,, at least 10 m²/g, at least 15 m²/g, at least 20 m²/g, at least 25 m²/g, at least 30 m²/g, or at least 35 m²/g). In some embodiments, the catalyst has a specific surface area of 40 m²/g or less, as measured using the BET method (e.g,, 35 m²/g or less, 30 m²/g or less, 25 m²/g or less, 20 m²/g or less, 15 m²/g or less, or 10 m²/g or less).

The catalyst can have a specific surface area ranging from any of the minimum values described above to any of the maximum values described above. In some embodiments, the catalyst has a specific surface area of from 5 m²/g to 40 m²/g, as measured using the BET method (e.g., from 10 m²/g to 40 m²/g, from 10 m²/g to 25 m²/g, from 10 m²/g to 20 m²/g, from 20 m²/g to 40 m²/g, or from 10 m²/g to 15 m²/g).

The catalysts can be disposed on a conductive support (e.g., the surface of an electrode, such as a copper electrode (e.g., copper foil) or carbon electrode) to form an electrode for use in conjunction with the methods described herein.

For some applications, including many catalytic applications, it may be of interest to deposit the catalysts described herein on a support, such as a carbonaceous support. Accordingly, also provided are compositions comprising a catalyst described herein deposited on a support, such as a carbonaceous support. The carbonaceous support may comprise any type of carbon that suitably supports the catalyst to provide a catalyst having suitable activity. The carbonaceous support can comprise an amorphous carbon, a crystalline or graphitic carbon, or a vitreous or glassy carbon. Also, the carbonaceous support can be in any suitable form (e.g., in the form of a powder, fiber, or flake), and can have any suitable crystallographic orientation, crystallite size, interlayer spacing, density, particle size, or particle shape. The carbonaceous support can comprise a carbon selected from Ketjen Black, carbon black, lamp black, acetylene black, mesocarbon, graphite, pyrolytic graphite, single-wall carbon nanotubes, multi-wall carbon nanotubes, Vulcan carbon, and carbon fiber. In some embodiments, the carbonaceous support has an average particle size of from 0.01 μm to 10 μm.

As disclosed herein, the systems disclosed for electrochemically producing hydrogen peroxide comprise at least one pair of electrodes, wherein each pair of electrodes comprises an anode and a cathode. In some embodiments, the at least one of the anode or the cathode is derived from the catalyst described herein. In some embodiments, the systems disclosed for electrochemically producing hydrogen peroxide comprise at least one pair of electrodes, wherein each pair of electrodes comprises a working electrode comprising the catalyst in electrochemical contact with a counter electrode.

Methods for preparing nanoporous electrode materials are described in International Publication No. WO 2016/054400 to Anne Co, the disclosure of which is incorporated herein by reference in its entirety.

The electrochemical reduction of oxygen or oxidation of water by catalysts may produce one or more products. For example, the electrochemical reduction of oxygen or oxidation of water may produce hydrogen peroxide, water, and oxygen. The catalysts for electrochemical reduction of oxygen or oxidation of water disclosed herein, however, are selective towards the formation of hydrogen peroxide. In some embodiments, the hydrogen peroxide is formed at a Faradaic efficiency of at least 10% (e.g., at least 15%, at least 20%, or at least 25%). In certain embodiment, the catalyst is selective for the formation of hydrogen peroxide over water, such that the hydrogen peroxide is formed with at least 10 times greater Faradaic efficiency than water. In certain examples, water is formed at a Faradaic efficiency of less than 0.5% (e.g., less than 0.1%, or less than 0.05%). In some embodiments, the system is configured to form hydrogen peroxide at a Faradaic efficiency of at least 10% (e.g., at least 15%, at least 20%, or at least 25%). In certain embodiment, the catalyst used in the system is selective for the formation of hydrogen peroxide over water, such that the hydrogen peroxide is formed with at least 10 times greater Faradaic efficiency than water. In certain examples, the system is configured to form water at a Faradaic efficiency of less than 0.5% (e.g., less than 0.1%, or less than 0.05%).

The electrodes (including the anode and cathode) comprising the catalyst for electrochemically producing hydrogen peroxide, in some embodiments, is affixed to or otherwise embedded in a substrate. In some embodiments, the electrodes is printed on a substrate using, for example, conductive printing techniques. For example, the electrodes can be printed on the substrate using screen-printing techniques, using a (conductive) ink-jet printer, and the like. It is to be appreciated that any other deposition or incorporation methods may be used to form the at least one pair of electrodes on or within the substrate.

The substrate comprising the electrodes in the invention systems disclosed herein for electrochemical production of hydrogen peroxide can be made of any material. The substrate is preferably a biocompatible substrate. The term “biocompatible”, as used herein, refers to having the property of being biologically compatible by not producing a toxic, injurious, or immunological response in living tissue.

In some embodiments, the substrate is a fabric substrate. The term “fabric” as used herein refers to any kind of material, including woven materials, nonwoven materials, knitted or pleating (plaited) material, a scrim or entangled fibers, filaments and/or any other kind of materials formed from yarn.

In some embodiments, the fabric substrates described herein, do not include paper. Paper, as used herein, refers to cellulose-based sheet materials used for writing, printing, packaging, and other applications. Paper is generally produced by pulping a cellulosic material to form a pulp containing cellulosic fibers, amalgamating the cellulosic fibers to form a wet web, and drying the wet web. In the finished paper, the fibers are held together by mechanical interlocking and hydrogen bonding.

The fabric substrate can be made of any natural or synthetic materials. The fabric substrate, however, is generally non-conductive when dry, but made of materials that are generally absorbent or at least wicking (e.g., silk, cotton, hemp, bamboo, cellulose, poly microfiber-based fabrics, flax, wool, ramie, lyocell, polyester, nylon, down, fur, jute, rubber, PVC, soy, etc.). Generally, in regard to the fabric substrate, it is comprised of a material that is substantially electrically insulating. For example, the fabric substrate may be comprised of silk, cotton, polyester, and the like. At least one of the anodes and the cathodes of the electrodes can be printed on, affixed to, or otherwise embedded in a fabric substrate. In some embodiments of the systems disclosed herein for electrochemical production of hydrogen peroxide, at least one of the anodes and the cathodes is woven into the fabric substrate.

In some embodiments, the fabric substrate comprising the electrodes is in the form of a cleaning wipe. The cleaning wipe may be disposable, and primarily used in a single use modality, being made available for use in a resealable or single use container. Suitable containers include, for example, a canister, tub, tray, flexible pouch, packet, or the like. The flexible pouch may be, for example, resealable, be contained in an outer carton, may have a plastic lid, or any combinations thereof.

In some embodiments, the cleaning wipe disclosed herein comprises at least one pair of electrodes (including the anode and the cathode) printed or woven into the fabric substrate. In some instances, the cleaning wipe can be stored dry (i.e., in the absence of an aqueous fluid) or impregnated with the aqueous fluid in a ready to use fashion. The cleaning wipe may be used to clean clothes, fabric, carpet, and other inanimate surfaces, such as, for example, a countertop, drawer, shelf, and metal fixture. In some embodiments, the cleaning wipe is applicable to clean a wound.

In some embodiments, the wipe is made of an absorbent, or semi-absorbent material, or a biocompatible material. It may be in the form of a woven, nonwoven, or partially woven sheet. In some embodiments, the wipe, or wipe material is made of a substantially fibrous, or semi-fibrous material. The material may be cotton, nylon, polyester, polyethylene, polypropylene, porous foam, rayon, reticulated foam, reticulated thermoplastic film, thermoplastic scrim, wood pulp, or any combinations thereof.

In other embodiments, the wipe is single surfaced, or is incorporated a backing member. The backing member may be pervious or impervious to the cleaning solution. The backing member may be manufactured from a wide range of materials such as woven or non-woven material, polymeric material, natural fiber, synthetic fiber, or any combinations thereof. The backing member may provide structural support to the wipe, impart texture to the wipe, provide a prophylactic barrier, or any combinations thereof.

In some embodiments, the wipe material is used alone, or in combination with a binder such as lignin, starch, acrylic, vinyl acrylic, styrene-butadiene, or the like, and others known in the art. These binders may be used alone or in any combinations thereof.

The wipe may be prepared/produced from the wipe material by any process known to those skilled in the art. Such a process includes, but is not limited to carded/chemically or resin bonded, non-woven wipe carded/chemically or resin bonded, air laid chemically bonded, carded thermally bonded, airlaid thermally bonded, carded spunlaced or hydroentangled, wet laid chemically bonded, wet laid spunlaced or hydroentangled, meltblown, spunbond, apertured, needle punched, or any combinations thereof.

The wipe may also be smooth, textured, abrasive or any combinations thereof to aid in the ease of use by the end-user. In some embodiments, the wipe is a three-dimensional, macroscopically-expanded, fluid pervious web.

The wipe may be provided or placed into the container in any fashion or method known to those skilled in the art. By way of example, the wipe may have a C-fold, a half fold, a Z-fold, a modified Z-fold, perforated in a roll, perforated and folded in a stack, interleafed, cross folded, quarter folded, or any combinations thereof.

It is known in the art that the UV light (e.g., lights from the sun) causes degradation of hydrogen peroxide making the hydrogen peroxide containing solution or the wipe ineffective as a disinfection means. Thus, a hydrogen peroxide solution or the wipe therefrom cannot be stored in a clear container, which causes a storage problem. The in situ hydrogen peroxide producing wipe disclosed herein, on the other hand provides a solution of this long felt but unsolved needs to afford the disinfection means without worrying storage issue allowing the use of the wipe to disinfect the object (such as wounds) in situ when the invention cleaning wipe contacts the liquid containing surfaces of an object (e.g., an area of wounds), or applying power source to the liquid presoaked wipe, causing the production of hydrogen peroxide releasing into the fabric substrate of the wipe.

In some embodiments, the substrate comprising the at least one pair of electrodes serves as an external surface of the system following application to a tooth surface. For example, the substrate can be a material that provides the primary structural element for the system either during manufacture or during use. In some examples, the substrate may permit the systems disclosed herein to follow the contours of the teeth and be worn comfortably in the mouth without rubbing or otherwise irritating the lips or tongue. In some embodiments, the material used for the substrate is inert and translucent so that the system is unobtrusive when worn. In other embodiments, the material used for the substrate is inert and colored, so that the system is obtrusive when worn. Examples of materials useful for the substrate are polyesters, polyethylene, polypropylene, polyurethanes and polyether amides.

In some embodiments, the substrate is in the form of a patch. The term “patch,” as used herein, refers to a three-dimensional solid or semi-solid composition that can be adhered to teeth, or other hard structures in the mouth, such as dentures, by means of an adhesive portion or layer for example, which can contain the systems disclosed herein, and which can release an effective amount of hydrogen peroxide, for a desired period of time, from its site on the tooth into the oral cavity. In some embodiments, the patch optionally comprises an adhesive portion adjacent to the at least one pair of electrodes. The adhesive portion is used to adhere the other portion of the system to the tooth or other hard dental structure.

In some embodiments, the substrate that serves as an external surface of the system following application to a tooth surface is occlusive (i.e., not “breathable”), thus does not allow the hydrogen peroxide produced by the system to leak through the layer, and contact the mucous membranes of the mouth and gums. When ready for use, the substrate can be pre-moistened so that the electrochemical reaction is initiated as well as the tackiness is increased, and the substrate will adhere to the teeth.

One advantage of this embodiment is that the hydrogen peroxide cannot substantially leak out through the substrate and cause irritation in those individuals sensitive to the whitening agent or to any unpleasant flavor or sensation. Thus, this embodiment provides a solution of this long felt but unsolved needs to whitening or disinfecting teeth without irritation in those individuals sensitive to the whitening agent or to any unpleasant flavor or sensation, unlike the traditional known methods requiring immersing the substrate to a hydrogen peroxide solution or by other hydrogen peroxide application methods.

Alternatively, in some other embodiments, the substrate is fully or partially non-occlusive, and therefore can fully hydrate in situ, in position on the teeth.

As described herein, the electrochemical action of the systems disclosed for electrochemically producing hydrogen peroxide can be activated by an aqueous solution of an electrolyte. In some embodiments, the anode and the cathode comprising the catalyst are positioned on or into a substrate, wherein the substrate comprises an aqueous solution of an electrolyte in electrochemical contact with the anode and the cathode. Any suitable electrolyte can be used. For example, the electrolyte can be selected from any conductive liquid including a saline solution (i.e., sodium chloride in water), perspiration, wound exudate, saliva, or water. In some cases, electrochemical action will not begin until after the systems disclosed herein comes into contact with an electrolyte, such as wound exudate, sweat (aka perspiration), saliva, or a saline solution. Prior to that, the systems can be dry and may have a long shelf (aka storage) life which provides a solution of this long felt but unsolved needs of the unstable, hard to store of the hydrogen peroxide containing products.

In some embodiments, the aqueous solution can have a salinity of 2,500 ppm or greater (e.g., 3,000 ppm or greater, 3,500 ppm or greater, 4,000 ppm or greater, 4,500 ppm or greater, 5,000 ppm or greater, 5,500 ppm or greater, 6,000 ppm or greater, 6,500 ppm or greater, 7,000 ppm or greater, 7,500 ppm or greater, 8,000 ppm or greater, 8,500 ppm or greater, 9,000 ppm or greater, 9,500 ppm or greater, 10,000 ppm or greater, 10,500 ppm or greater, 11,000 ppm or greater, 12,000 ppm or greater, 14,000 ppm or greater, 15,000 ppm or greater, 18,000 ppm or greater, 20,000 ppm or greater, 22,000 ppm or greater, 25,000 ppm or greater, 28,000 ppm or greater, 30,000 ppm or greater, 35,000 ppm or greater, 40,000 ppm or greater, 45,000 ppm or greater, or 50,000 ppm or greater). The aqueous solution can have a salinity of 50,000 ppm or greater (e.g., 45,000 ppm or less, 40,000 ppm or less, 35,000 ppm or less, 30,000 ppm or less, 25,000 ppm or less, 22,000 ppm or less, 20,000 ppm or less, 18,000 ppm or less, 15,000 ppm or less, 12,500 ppm or less, 10,000 ppm or less, 9,500 ppm or less, 9,000 ppm or less, or 8,500 ppm or less). The salinity of the aqueous solution can range from any of the minimum values described above to any of the maximum values described above. For example, the salinity of the aqueous solution can be from 2,500 ppm to 50,000 ppm (e.g., from 3,000 ppm to 50,000 ppm, from 5,000 ppm to 50,000 ppm, from 6,000 ppm to 50,000 ppm, from 2,500 ppm to 30,000 ppm, from 6,000 ppm to 30,000 ppm, from 6,000 ppm to 25,000 ppm, from 6,500 ppm to 20,000 ppm, from 8,000 ppm to 20,000 ppm, from 2,500 ppm to 15,000 ppm, or from 8,500 ppm to 15,000 ppm).

When the substrate becomes moist, the moisture acts as an electrolyte to the electrodes, a redox reaction occurs between the cathode and anode of each cell, generating electrical energy. As previously noted, moisture may be derived from perspiration, wound exudate, body fluids including blood, saliva, and the like. If more than one set of electrodes are present, a flexible conductor electrically connects an anode or cathode of one cell to a cathode or anode, respectively, of another cell, so that the cells are electrically connected in series or parallel. Flexible connectors used to inter-connect different cells can be realized using conductive wires or traces. These may be implemented via conductive inks, conductive threads, conductive wires, and the like. These conductive inter-connections might be pre-printed on the substrate, followed by deposition of the anode and cathode materials and the hydrophobic barrier. Alternatively, the anode and cathode materials. In some embodiments, a hydrophobic barrier (e.g., hydrophobic sprays, lubricant impregnated surfaces, carbon nanotubes, silicone, etc.) is located between each cell to block moisture migration between the cells. In some embodiments, a substrate (or layers of substrate “sandwiched” together) is pre-soaked with a strong electrolyte and further used to moisten the cells in the form of an underlying electrolyte “cushion”.

In some embodiments, the systems disclosed herein comprise a power source. The power source can be a battery cell comprised of at least one set of source electrodes on or embedded within a substrate to realize a source cathode (positive terminal) and a source anode (negative terminal) of the cell. When the substrate having the source electrodes thereon or embedded within comes into contact with a conductive liquid (e.g., sweat, wound exudate, fluids, etc.), the latter acts as an electrolyte, causing the source anode to oxidize, and the battery cell to generate DC power when connected to the circuitry of the system for producing hydrogen peroxide described herein. For example, in one of the embodiments the power source may comprise oxides of silver (Ag₂O) and zinc (Zn) at the source cathode and source anode, respectively. In this particular case, when the source cathode interacts with the conductive liquid, OH⁻ ions are generated. These OH⁻ ions then migrate to the source anode and are consumed. In this way, DC voltage and current are generated just by getting the electrochemical power source moistened via a conductive liquid.

In the above example, the source electrodes, in some embodiments, comprise silver oxide (Ag₂O) and zinc (Zn), though it is to be appreciated that the electrodes can be comprised of any materials that undergo a reduction-oxidation process that generates electrical energy in the presence of an electrolyte. Generally, the source anode and the source cathode are comprised of biocompatible electrically-conductive materials. Non-limiting examples of other materials that may be used for the source electrodes include silver, silver chloride, silver compounds, gold, gold compounds, platinum, platinum compounds, and/or binary alloys of platinum, cobalt or palladium with phosphorus, or binary alloys of platinum, nickel, cobalt or palladium with boron, cadmium, lithium, aluminum, iridium, mixed metal oxides, metal phosphates, metal nanoparticles, and the like. Non-metallic materials are also contemplated for source electrode formation such as conductive polymers and the like. Conductive polymers can include, but are not limited to, polyaniline, polythiophene, polypyrrole, polyphenylene, poly(phenylenevinylene), and the like.

In some embodiments, the power source in the systems comprise an inter-connection of several of the aforementioned cells in order to boost/scale the generated DC power levels. For example, the systems disclosed herein may comprise a plurality of cells, wherein the plurality of cells are connected using flexible connectors either in electrical series and/or electrical parallel to increase a voltage and/or a current of the generated electrical energy. Generally, each of the plurality of cells is separated from an adjoining cell by a hydrophobic barrier.

In certain embodiments, the systems disclosed herein specifically do not include a power source, which can further extend the storage time of the systems. For example, battery-less systems for electrochemically producing hydrogen peroxide are disclosed herein. In these embodiments, the electrochemistry of the systems is used to power the systems. In certain embodiments, a low resistivity flexible connector is used to connect the anode and cathode in these embodiments.

As disclosed herein, the power level of the disclosed system for generating hydrogen peroxide varies based on the design of the system. The amount of hydrogen peroxide generated is dependent on the amount of current passing through and length of time the system is operated. In some embodiments, the speed of hydrogen peroxide generation can be controlled through the number of batteries and the time the system runs can be controlled by adjusting battery capacity. In some instances, one battery (1 Volt) draws 0.1 mA and delivers 0.52 μM hydrogen peroxide per second. In some instances, one battery may draw 1 mA for 5 min and generate 5 μmol hydrogen peroxide. In some embodiments, the current applied to the electrodes can be 0.5 mA or greater (e.g., 1 mA or greater, 1.5 mA or greater, 2 mA or greater, 2.5 mA or greater, 3 mA or greater, 3.5 mA or greater, or 5 mA or greater). The applied current can be 25 mA or less (e.g., 20 mA or less, 15 mA or less, 10 mA or less, 7.5 mA or less, 5 mA or less, 4 mA or less, 3 mA or less, 2 mA or less, 1 mA or less, or 0.9 mA or less). The applied current can range from any of the minimum values described above to any of the maximum values described above. For example, the applied current can be from 0.5 to 5 mA (e.g., from 0.5 to 25 mA, from 1 to 25 mA, or from 1 to 15 mA).

The amount of hydrogen peroxide generated by the systems disclosed herein can be 1 μmol or greater (e.g., 1 μmol or greater, 5 μmol or greater, 10 μmol or greater, 20 μmol or greater, 50 μmol or greater, 100 μmol or greater, 500 μmol or greater, 750 μmol or greater, 1 mmol or greater, 2 mmol or greater, 5 mmol or greater, 10 mmol or greater, 20 mmol or greater, 50 mmol or greater, 100 mmol or greater, 200 mmol or greater, 300 mmol or greater, 500 mmol or greater, 750 mmol or greater, or 1 mol or greater). The amount of hydrogen peroxide generated by the systems disclosed herein can be 1 mol or less (e.g., 900 mmol or less, 800 mmol or less, 750 mmol or less, 700 mmol or less, 600 mmol or less, 500 mmol or less, 400 mmol or less, 350 mmol or less, 250 mmol or less, 200 mmol or less, 100 mmol or less, 50 mmol or less, 20 mmol or less, 1 mmol or less, 1 mmol or less, 900 μmol or less, 750 μmol or less, 600 μmol or less, 500 μmol or less, 250 μmol or less, 100 μmol or less, 50 μmol or less, 20 μmol or less, 10 μmol or less, or 5 μmol or less). The amount of hydrogen peroxide generated by the systems can range from any of the minimum values described above to any of the maximum values described above. For example, the amount of hydrogen peroxide generated by the systems can be from 0.5 μmol to 1 mol, (e.g., from 1 μmol to 1 mol, from 100 μmol to 500 mmol, from 100 μmol to 100 mmol, or from 1 μmol to 1 mmol).

In some embodiments, the amount of hydrogen peroxide generated by the systems disclosed herein can be 0.2% by weight or greater (e.g., 0.5% by weight or greater, 0.8% by weight or greater, 1% by weight or greater, 1.5% by weight or greater, 2% by weight or greater, 2.5% by weight or greater, 3% by weight or greater, 3.5% by weight or greater, 4% by weight or greater, 4.5% by weight or greater, 5% by weight or greater, 5.5% by weight or greater, 6% by weight or greater, 6.5% by weight or greater, 7% by weight or greater, 7.5% by weight or greater, 8% by weight or greater, 8.5% by weight or greater, 9% by weight or greater, 9.5% by weight or greater, or 10% by weight or greater), based on the weight of the aqueous fluid in the systems. The amount of hydrogen peroxide generated by the systems disclosed herein can be 20% by weight or less (e.g., 18% by weight or less, 15% by weight or less, 12% by weight or less, 10% by weight or less, 9% by weight or less, 8.5% by weight or less, 8% by weight or less, 7.5% by weight or less, 7% by weight or less, 6.5% by weight or less, 6% by weight or less, 5.5% by weight or less, 5% by weight or less, 4.5% by weight or less, 4% by weight or less, 3.5% by weight or less, 3% by weight or less, 2.5% by weight or less, 2% by weight or less, or 1.5% by weight or less), based on the weight of the aqueous fluid in the systems. The amount of hydrogen peroxide generated by the systems can range from any of the minimum values described above to any of the maximum values described above. For example, the amount of hydrogen peroxide generated by the systems can be from 0.5% to 25% by weight, (e.g., from 0.5% to 20% by weight, from 1% to 15% by weight, from 1% to 10% by weight, from 1% to 5% by weight, or from 2% to 5% by weight,), based on the weight of the aqueous fluid in the systems.

### Exemplary Methods of Applications of the Systems Disclosed Herein

Exemplary methods of using the systems disclosed herein are provided. In some embodiments, the methods of treating or preventing a bacterial infection in a wound by the system disclosed herein comprise applying a hydrogen peroxide generating biocompatible substrate to the wound, wherein the hydrogen peroxide generating biocompatible substrate comprises a biocompatible substrate; and at least one pair of electrodes positioned on or within the biocompatible substrate, and allowing a therapeutically effective amount of hydrogen peroxide generated from the hydrogen peroxide generating biocompatible substrate to contact the wound. In some embodiments, the systems are used to inhibit or disrupt bacterial growth in the wound or inhibit or disrupt biofilm in the wound. In certain embodiments, the hydrogen peroxide generating biocompatible substrate is wetted with an aqueous liquid prior to applying to the wound or may be dry prior to applying to the wound.

Tooth whitening methods are also provided herein. In some embodiments provide a method of applying a tooth whitening system disclosed herein to a tooth, wherein the tooth whitening system comprises a substrate that serves as an external surface of the tooth whitening system following application to a tooth surface; and at least one pair of electrodes positioned on or within the substrate, and allowing an effective amount of hydrogen peroxide generated from the tooth whitening system to contact the tooth.

Methods of removing stain from a surface are further provided. In some embodiments provide a method of applying a hydrogen peroxide generating cleaning wipe disclosed herein to a surface, wherein the hydrogen peroxide generating cleaning wipe comprises a substrate; and at least one pair of electrodes positioned on or within the substrate, and allowing an effective amount of hydrogen peroxide generated from the substrate to contact the stain, thereby removing the stain from the material.

### Kits

Kits comprising the hydrogen peroxide generating systems disclosed herein are provided. In some embodiments provide a kit comprising one or more of the hydrogen peroxide generating systems described herein in a container. Suitable containers include, for example, a canister, tub, tray, flexible pouch, and packet. The container may be, for example, resealable, be contained in an outer carton, may have a plastic lid, or any combinations thereof.

### EXAMPLES

### Example 1: Methods of Preparation of Exemplary Systems

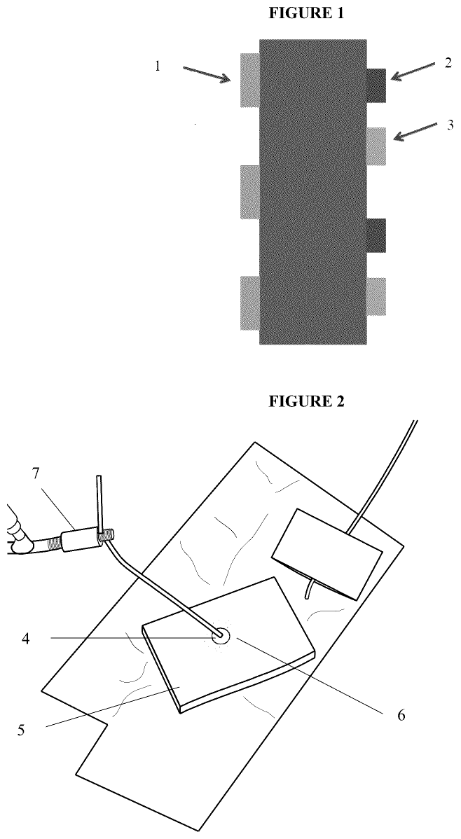

FIG. 1 provides an exemplary schematic diagram showing an integrated self-powered peroxide generating fabric, comprising a catalyst 1, anode 2, cathode 3. Such fabric is applicable as illustrated in FIG. 2 showing an exemplary printed catalyst (e.g., printed Cu dot 4) on a soaked fabric (e.g., permanganate dyed fabric 5) generating peroxide (white region around dot 6) when powered (e.g., by a current source 7). The amount of peroxide generated is dependent on the current and time run (e.g., 1 mA for 5 minutes generates 5 μmol on one dot).

A nanoporous copper electrode (0.221 cm²) is made from 10% polyvinylidene fluoride (PVDF) mixed with n-methyl-2-pyrrolidone (NMP), with 90% copper. The wet catalyst was added to a polyester fabric and dried in an oven for 30 min at 90° C. A Ni or Pt wire (0.1 mm dia, Alfa Aesar)) was woven into the fabric as a contact.

### Catalyst Oxidation and Reduction Shift Data

A current of 0.1 mA and 0.05 mA was applied to the copper catalysts to determine the voltage required when hydrogen peroxide is produced by an oxidation process and then by a reduction process. These tests used a potentiostat (Gamry Reference 600). The working electrode is the nanoporous copper, counter electrode is a Zn (2.5 cm ×0.5 cm) electrode, and the reference electrode was a platinum pseudo reference. The electrolytes used were Artificial Sweat (ISO-3160-2, Reagents), Dulbecco phosphate saline buffer (PBS, ThermoFisher Scientific), 0.35M NaCl and PBS+0.35M NaCl.

Experiments were performed as shown in Table 1, which shows oxidation of water has smaller voltage difference (less power) than reduction of oxygen using a nanoporous copper catalyst. The equations for oxidation and reduction shifts are shown in FIG. 3A/B. FIG. 3A is a graph showing how far the voltage shifts from OCP on application of positive and negative currents. FIG. 3B shows peroxide can be generated through oxidation or reduction using a copper catalyst. The determined oxidation (current out of Cu) will require less power for the same generated peroxide.

TABLE 1
Catalyst oxidation and reduction shift data.

| | | | | | | Artifi- | |
|---|---|---|---|---|---|---|---|
| | | | | | | cial | |
| | | | | PBS | | Sweat | |
| | | | | mim- | | con- | |
| | | | | icking | PBS + | taining | |
| | | | 0.35M | body | 0.35M | 0.35M | |
| | | | NaCl | fluids | NaCl | NaCl, | 10M |
| | Current | | (ΔV) | (ΔV) | (ΔV) | (ΔV) | NaOH |
| Oxidation | 0.05 | mA | 0.259 | 0.261 | 0.349 | 0.072 | 0.286 |
| Oxidation | 0.1 | mA | 0.33 | 0.306 | 0.417 | 0.104 | 0.05 |
| Reduction | −0.05 | mA | 0.75 | 0.8201 | 0.69 | 0.1968 | 0.458 |
| Reduction | −0.1 | mA | 0.82 | 0.9267 | 0.769 | 0.232 | 0.278 |

### Fabric Battery

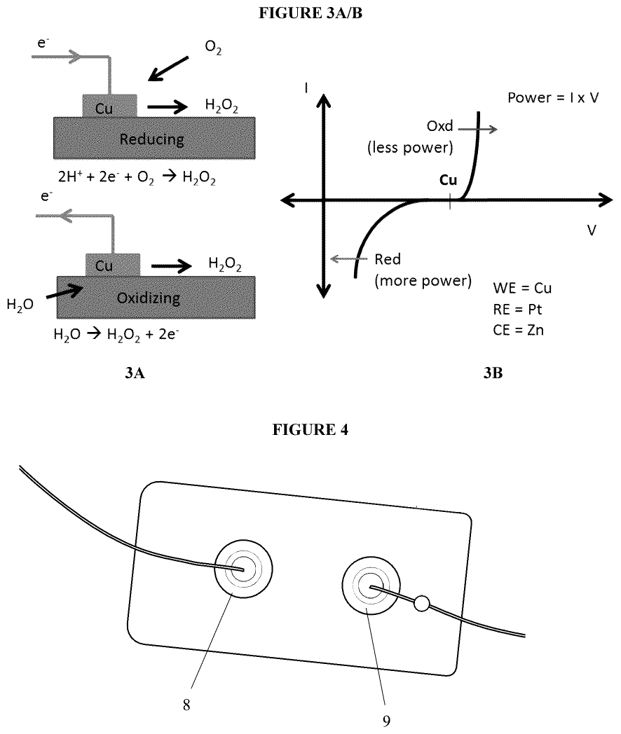

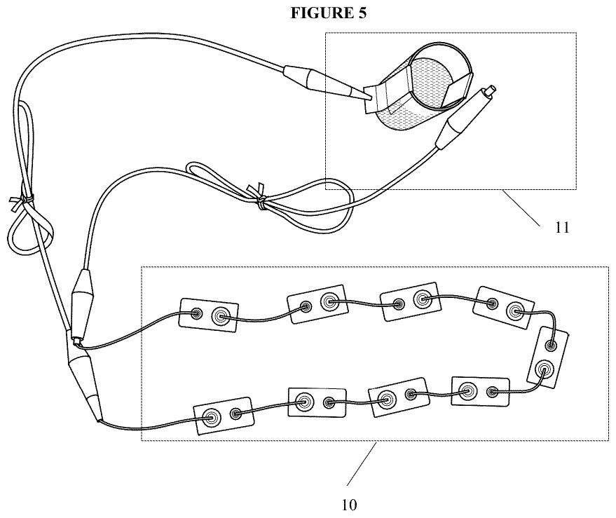

An exemplary fabric battery was constructed using a Zn electrode 8 (10% PVDF +NMP, and 90% Zn) and an Ag₂O electrode 9 (10% PVDF+NMP, and 90% Ag₂O) as shown in FIG. 4 printed with an area of 0.25 cm². Ni or Pt wire is used as the contact wire. Open circuit potential is around 1.0 V in saltwater (0.5 M NaCl) and PBS, and OCP was close to 1.5 V in 10 M NaOH as an electrolyte.

To determine the experimental capacity, batteries were discharged at C/8 (820 μA) using different electrolytes, 0.35 M NaCl (mimicking sweat), PBS (mimicking body fluids), and 10 M NaOH. Table 2 shows fabric batteries in NaCl or PBS buffer has comparable capacity to a 10 M NaOH electrolyte used in conventional alkaline batteries.

TABLE 2
Comparison of fabric batteries in NaCl or buffer.

| | | Body Fluid, PBS + | |
|---|---|---|---|
| | Sweat, 0.5M NaCl | 0.5M NaCl | NaOH |
| Voltage @ OCP (V) | 0.96 | 0.97 | 1.46 |
| Voltage @ 0.29 | 0.83 | 0.81 | 0.81 |
| mAhr | | | |
| Capacity (mAhr) @ | 0.37 | 0.46 | 0.37 |
| 100 μA drain | | | |

### Scalability

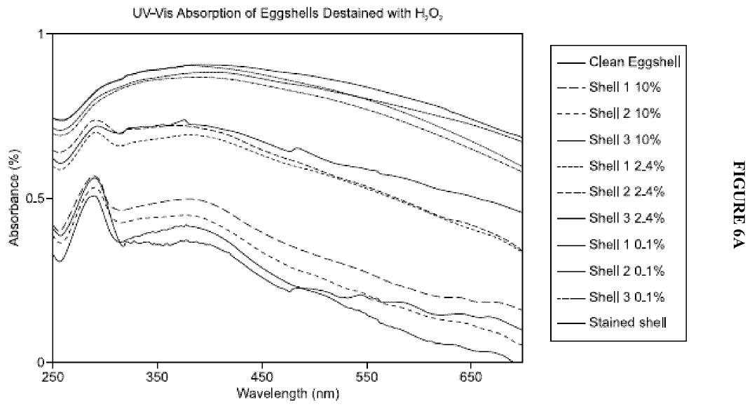

Table 3 shows the speed of hydrogen peroxide generation can be controlled through the number of batteries. Time runs can be controlled by adjusting battery capacity. FIG. 5 shows a system with a plurality of batteries connected in series (e.g., fabric cells 10). The speed of hydrogen peroxide generation (as illustrated as a peroxide generator 11 in FIG. 5) can be controlled through a number of batteries. The time the device runs can be controlled by adjusting battery capacity. One battery (1 Volts) draws 0.1 mA and delivers 0.52 μM/sec, which is safe for human use.

TABLE 3
Scalability of fabric batteries at OCP

| # of Cells | Sweat (V) | Body Fluid (V) | NaOH (V) |
|---|---|---|---|
| 1 | 0.96 | 0.97 | 1.46 |
| 2 | 1.52 | 1.76 | 2.54 |
| 3 | 2.07 | 2.41 | 2.85 |

### Teeth Whitening Effect by the Systems

To test the effects of the hydrogen peroxide on teeth whitening, a white eggshell substitute was first dyed with a solution of coffee before being placed on the catalyst electrode immersed in 0.5M NaCl. Hydrogen Peroxide was generated at 10%, 2.5%, 0.1%, as typical of teeth-whitening products. Eggshells were then crushed and put into a UV-vis spectroscopy.

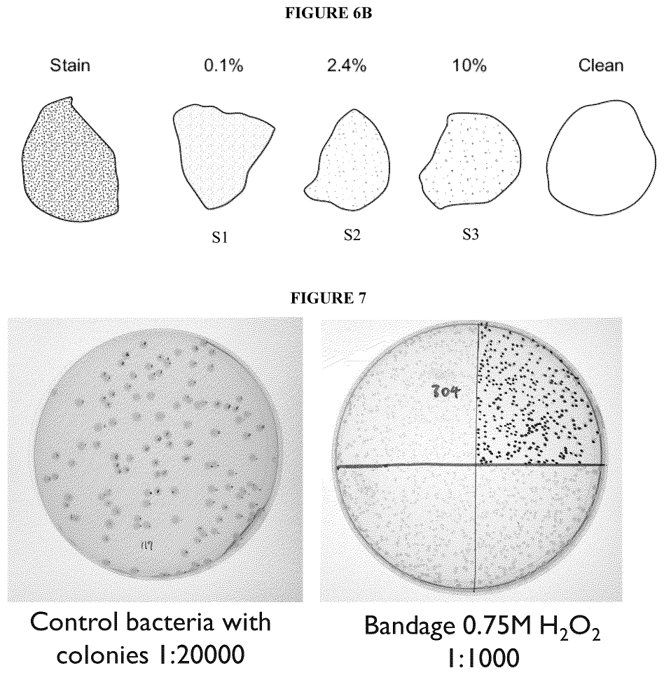

FIG. 6A provides a figure (UV-vis absorbance (%) vs UV wavelength (nm)) showing the results of hydrogen peroxide treatments of the coffee stained eggshells in different concentrations equivalent to commercial products (S1 (0.1%), S2 (2.4%), S3 (10%)). FIG. 6B illustrates a graph showing UV-VIS of crushed eggshells with CaCO₃ background (R %). The lighter eggshells are more reflective compared to darker eggshells.

### Disinfecting and Wound Healing

Hydrogen Peroxide Inhibition Test: Growth Inhibition was tested by disk diffusion test. A plate with Lysogeny Broth (LB) Agar media was evenly seeded with a 1 Optical Density (OD) culture of Escherichia coli (strain BW25113). Paper filter disks were placed on the surface of inoculated agar and were moistened with 0.75 M of generated hydrogen peroxide from the catalyst in PBS. The same procedure was repeated for a PBS solution only, without the generated hydrogen peroxide, as a control. Plates were left to dry and incubated at 37° C. for 18-24 hr.

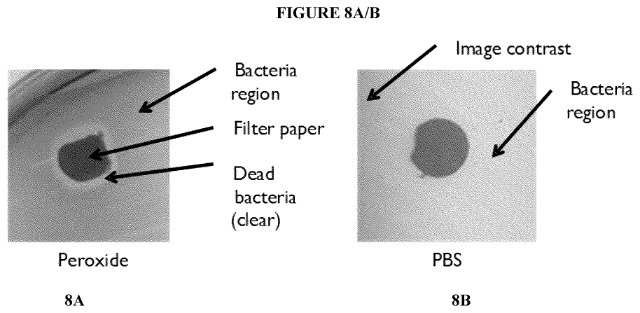

The results of the inhibition test are shown in FIG. 8A/B. The clear zone shown around the moistened disks with 0.75 M H₂O₂ is the zone of growth inhibition (see 8A), whereas the moistened disks with 1×PBS showed no zone of inhibition (i.e., no clear zone in 8B). This test is only qualitative and does not necessarily indicate that the bacteria has been killed, but prevented from growing by the hydrogen peroxide.

Killing (Antimicrobial) Test: To determinate the percentage of bacteria killed by the hydrogen peroxide, we grew an E. coli culture from a single colony on LB media at 37° C. until 0.05 OD is measured. The initial colony forming units per mL (CFU/mL) were counted by plating 50 uL of a dilution 1:10,000 of the culture on LB agar plates. Next, 3 ml of the bacterial culture were treated with 1 ml of separately generated H₂O₂ 0.75 M such that the final concentration of H₂O₂ in the bacteria containing plates reached around 0.25 M. To count the bacterial survival after treatment, 100 μl of 1:10,000 dilution were plated on LB agar plates and incubated at 37° C. for 18-24 hrs. The percentage of killed bacteria was calculated using the initial CFU as 100% and subtracting the recovered CFU after treatment. Table 4 shows the results of the antimicrobial test by triplicate experiments, which indicates that the bacteria population can be killed at a rate of 2.5% CFU/μmol H₂O₂. Agar plates containing bacteria from this test can be found in FIG. 7.

TABLE 4
E. Coli population.

| Control (CFU x 10⁶) | Peroxide Treated (CFU x 10⁶) | % Killed (%) |
|---|---|---|
| 31.2 | 1.2 | 96.1 |
| 16.3 | 0.7 | 96 |
| 19.7 | 1.2 | 94.2 |

Killing Bacteria by Electric Current:

It was determined that 5 mA of electric current applied for 30 min to a 0.05 OD of E. coli culture can kill up to 99.6% of bacteria. To test this an E. coli culture was grown from a single colony in LB media at 37° C., until reaching 0.05 OD. Next, 0.5 mL of this culture was treated with 5 mA of electric current for 30 min. The counting of CFU/ml before and after applying the current for 30 min was done by plating 50 and 100 μL of 1:10,000 dilution respectively on LB agar plates and incubating at 37° C. for 18-24 hrs. Table 5 show results by triplicate experiments which shows 99.6% of E. coli population was killed with hydrogen peroxide generated at 5 mA for 30 min.

TABLE 5
E. Coli population.

| Control (CFU x 10⁶) | Peroxide Treated (CFU x 10⁶) | % Killed (%) |
|---|---|---|
| 14 | 0.05 | 99.6 |
| 99.6 | 0.04 | 99.6 |
| 48.4 | 0.015 | 99.6 |

### Conductivity of Various Media

The conductivity of several media was investigated. Table 6 shows bacteria medium has comparable conductivity to sweat and body fluid.

TABLE 6
Conductivity

| Liquid | Conductivity (mS/cm) |
|---|---|
| Bacteria media (LB broth) | 21.87 |
| Body Fluid (1% PBS) | 16.64 |
| Sweat (0.85% NaCl) | 16.25 |

Summary: the flexible materials/systems exemplified herein comprise a hydrogen peroxide producing electrode (oxidation efficient) and a battery. The exemplary systems can immediately generate any desired amount of hydrogen peroxide controllable through current (# of batteries) and time (capacity). H₂O₂ concentration can be adjusted to a 1-10% range for de-staining or tuned to lower concentrations for disinfecting and wound care.

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods in addition to those shown and described herein are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A flexible material comprising:
a. a substrate; and
b. at least one pair of electrodes positioned on or within the substrate, wherein each pair of electrodes comprises an anode and a cathode; and
wherein at least one of the anode and the cathode comprise a catalyst for the formation of hydrogen peroxide (H₂O₂) via an electrochemical reaction, wherein at least one of the anode and the cathode are woven into the substrate or are printed on the substrate.

2. The flexible material of claim 1, wherein the catalyst comprises nanoparticles having an average particle size of from 10 nm to 500 nm, as determined by scanning electron microscopy (SEM).

3. The flexible material of claim 1, wherein the catalyst comprises a nanoporous Cu catalyst, a nanoporous Cu-M catalyst, or a combination thereof, where M is a metal selected from the group consisting of Pt, Ir, Pd, Ag, Au, Rh, Ru, Zn, Sn, Ni, Fe, Re, Ga, In, Cd, Tl, and Ti.

4. The flexible material of claim 4, wherein the catalyst comprises a nanoporous Cu catalyst.

5. The flexible material of claim 1, wherein the catalyst is selective for the formation of hydrogen peroxide over water, such that the hydrogen peroxide is formed with at least 10 times greater Faradaic efficiency than water.

6. The flexible material of claim 1, wherein the substrate is selected from the group consisting of polyester, denim, cotton, flax, wool, hemp, ramie, silk, lyocell, nylon, down, fur, jute, rubber, PVC, bamboo, soy, ramie, rayon, viscose, linen, tencel, and blends thereof.

7. The flexible material of claim 1, wherein the substrate further comprises an aqueous fluid in fluid contact with the at least one pair of electrodes.

8. The flexible material of claim 7, wherein the aqueous fluid comprises any conductive liquid comprising perspiration, wound exudate, saline, saliva, or water.

9. The flexible material of claim 1, further comprising a power source having an output electrically coupled to the at least one electrode pair, wherein the power source comprises one or more cells.

10. The flexible material of claim 9, wherein the power source applies an electric current of 1 mA or greater.

11. An article comprising the flexible material of claim 1, wherein the article is a cleaning wipe, a wound care system, or a tooth whitening system.

12. The wound care system of claim 11, wherein the substrate is a biocompatible fabric substrate.

13. The wound care system of claim 12, wherein the biocompatible fabric substrate is a woven fabric.

14. The wound care system of claim 13, wherein the biocompatible fabric substrate further comprises an aqueous fluid in fluid contact with the at least one pair of electrodes.

15. The wound care system of claim 14, wherein the aqueous fluid comprises wound exudate.

16. The wound care system of claim 12, further comprising a power source having an output electrically coupled to at least one electrode pair, wherein the power source comprises one or more cells.

17. A method of treating or preventing a bacterial infection in a wound comprising:
applying the wound care system of claim 12 to the wound, and
allowing a therapeutically effective amount of hydrogen peroxide generated from the peroxide generating biocompatible substrate to contact the wound.

18. The method of claim 17, wherein the method is used to inhibit or disrupt bacterial growth in the wound; or inhibit or disrupt biofilm in the wound.

19. The method of claim 17, wherein the peroxide generating biocompatible substrate is wetted with an aqueous liquid prior to applying to the wound.

20. The cleaning wipe of claim 11, wherein the substrate is a fabric substrate which is applied onto a surface.

21. The cleaning wipe of claim 20, wherein the fabric substrate is produced from a process selected from the group consisting of carded/chemically or resin bonded, non-woven wipe carded/chemically or resin bonded, air laid chemically bonded, carded thermally bonded, airlaid thermally bonded, carded spunlaced or hydroentangled, wet laid chemically bonded, wet laid spunlaced or hydroentangled, meltblown, spunbonded, apertured, needle punched, and any combinations thereof.

22. The cleaning wipe of claim 20, wherein the fabric substrate is formed from a material selected from the group consisting of fiber, porous foam, reticulated foam, reticulated thermoplastic film, thermoplastic scrim, and combinations thereof.

23. The cleaning wipe of claim 22, wherein the fiber is selected from the group consisting of polyester, rayon, nylon, polypropylene, polyethylene, cotton, wood pulp, denim, flax, wool, hemp, ramie, silk, lyocell, down, fur, jute, rubber, PVC, bamboo, soy, ramie, viscose, linen, tencel, and blends thereof.

24. The cleaning wipe of claim 23, wherein the fiber is woven.

25. The cleaning wipe of claim 23, wherein the wipe further comprises at least one binder.

26. The cleaning wipe of claim 20, wherein the fabric substrate further comprises an aqueous fluid in fluid contact with the at least one pair of electrodes.

27. The cleaning wipe of claim 20, further comprising a power source having an output electrically coupled to at least one electrode pair, wherein the power source comprises one or more cells.

28. A method of removing stain from a surface, the method comprising:
applying the cleaning wipe of claim 20 and allowing an effective amount of hydrogen peroxide generated from the fabric substrate to contact a stain, thereby removing the stain from the surface.

29. The tooth whitening system of claim 11, wherein the substrate is applied on a tooth surface.

30. The tooth whitening system of claim 29, further comprising a tooth contact adhesive layer adhered to the fabric substrate.

31. The tooth whitening system of claim 29, wherein the substrate comprises a material selected from the group consisting of polyester, polyethylene, polypropylene, polyurethane, polyether amide, and blends thereof.

32. The tooth whitening system of claim 29, wherein the substrate is occlusive.

33. The tooth whitening system of claim 29, wherein the substrate further comprises an aqueous fluid in fluid contact with the at least one pair of electrodes.

34. The tooth whitening system of claim 29, wherein the aqueous fluid comprises saline, saliva, or water.

35. The tooth whitening system of claim 29, further comprising a power source having an output electrically coupled to at least one electrode pair, wherein the power source comprises one or more cells.

36. A tooth whitening method comprising:
applying the tooth whitening system of claim 29, and
allowing an effective amount of hydrogen peroxide generated from the tooth whitening system to contact the tooth.
